(19) United States
(12) Patent Application Publication
(10) Pub. No.: US 2021/0114023 A1
(43) Pub. Date: Apr. 22, 2021

(54) DETECTION SYSTEM AND METHOD FOR PRODUCING SAME

(71) Applicant: TECHNISCHE UNIVERSITÄT DARMSTADT, Darmstadt (DE)

(72) Inventors: Mario EL KHOURY, Darmstadt (DE); Wolfgang ENSINGER, Münster-Altheim (DE); H. Ulrich GÖRINGER, Roßdorf (DE); Sebastian QUEDNAU, Darmstadt (DE); Ivana DUZNOVIC, Mainz (DE); Helmut F. SCHLAAK, Ober-Ramstadt (DE)

(73) Assignees: TECHNISCHE UNIVERSITÄT DARMSTADT, Darmstadt (DE); TECHNISCHE UNIVERSITÄT DARMSTADT, Darmstadt (DE)

(21) Appl. No.: 16/627,181

(22) PCT Filed: Jun. 12, 2018
(86) PCT No.: PCT/EP2018/065542

§ 371 Date: Dec. 27, 2019

(30) Foreign Application Priority Data
Jun. 28, 2017 (DE) 10 2017 114 349.1

(51) Int. Cl. B01L 3/00; B81B 1/00; B81C 1/00; G01N 33/487; G01N 33/543; G01N 33/574; G01N 27/447; G01N 15/06

(52) U.S. Cl. CPC B01L 3/502707; B01L 2300/0645; B81C 1/00119; B81C 1/00206; G01N 33/48721; G01N 33/5438; G01N 33/57434; G01N 27/44739; G01N 27/44791; G01N 15/0656; B81B 2201/05; B81B 2203/0338; B81B 2203/04; B01L 2200/12; B81B 1/006

(57) ABSTRACT

A method for producing a detection system for biomolecules in a medium involves providing a first detector section having a first channel region and a second detector section having a second channel region. A membrane having at least one pore is provided and the first detector section and the second detector section are arranged on opposite sides of the membrane, such that at least part of the first channel region and the second channel region are separated by the membrane and the first channel region and the second channel region are connected to each another to form a channel system, in order to form a flow path for the medium through the at least one pore of the membrane. Along the flow path, through the membrane, bioreceptors are bound and/or coupled to the membrane in order to determine a concentration of the biomolecules in the medium by means of a measurement of the flow along the flow path.

Exemplary embodiments of the present invention relate to a detection system and a method for its production and more particularly to the detection of (bio) molecules (analytes, ligands, etc.) in abiotic and biotic systems.

### BACKGROUND OF THE INVENTION

Despite enormous biomedical research efforts, cancer still has high mortality rates. In addition, each type of cancer represents a therapeutic and diagnostic challenge for the treating physician. Unfortunately, reliable predictions of the extent to which the course of a disease will develop in the context of the therapeutic measures used are still fraught with high error rates. In this context, early diagnostic measurement methods are of great importance.

For example, prostate cancer (PCa) is the most common cancer in men in the Western world. In Germany alone, around 63,000 new cases are diagnosed each year. Fast on-site diagnostics that precisely produce a meaningful result should significantly increase the chances of a cure for PCa patients and, in many cases, be life-saving. The concentration of the prostate-specific antigen (PSA) can be measured for the early detection of PCa. This is a glycosylated protein that can be detected in the blood serum. In routine operation, the detection of PSA primarily uses antibody-based detection methods, which in addition to high costs often also produce false positive results. This fact is the starting point for the search for alternative measurement methods for the detection of PSA.

One approach is based on nucleic acid biopolymers such as aptamers, which are able to specifically recognize PSA and bind with high affinity. They can be used in a handy sensor system to measure the PSA concentration in the blood and thus to detect a possible prostate carcinoma. If such aptamers are fixed to the inner wall of nanoscale pores/channels of a filter film, a simple sensor for PSA detection can be produced.

An example of this type of detection can be found in the publication: Ali M, Nasir S, Ensinger W.: “Bioconjugation-induced ionic current rectification in aptamer-modified single cylindrical Pores”; Chem Commun 2015, 51: 3454-3459. A potential difference is created between both sides of a plastic film in order to generate a measurable ion current. If the blood serum contains the biomolecules to be detected, these molecules are bound to the aptamers in the pores, which leads to a narrowing of the cross-sectional area of the pores. This increases the electrical resistance of the individual pores depending on the concentration of the aptamer complexes. Consequently, the concentration of the biomolecule can be directly deduced from the decrease in the measured ion current.

In the production of these sensors, the aptamers used are applied to a multipore film, particularly in the area of the pores. This step is referred to as functionalization, since the resulting film is thereby predetermined for a specific function (detection of a biomolecule). In the production method used to date, the film was first functionalized, then cut back and finally arranged in a desired detection area. This so-called “pick-and-place” process is complex and can only be automated to a limited extent. Functionalized membranes can be damaged during the integration process (installation) and thus lose their functionality again. The functionalization of the etched membrane after its integration therefore offers advantages.

There is therefore a need for an improved production process for these sensors. There is also a need for improved aptamers that are highly sensitive to PSA and thus significantly improve the results.

### SUMMARY OF THE INVENTION

Exemplary embodiments of the present invention relate to a method for producing a detection system for biomolecules in a medium. The method comprises the following steps:
Providing a first detector section with a first channel region and a second detector section with a second channel region;
Providing a membrane with at least one pore; and
Arranging the first detector section and the second detector section on opposite sides of the membranes, so that at least part of the first channel region and the second channel region are separated by the membranes and the first channel region and the second channel region are connected to each other to form a channel system to form a flow path for the medium through the at least one pore of the membrane,

wherein along the flow path through the membranes, bioreceptors are formed on the membrane (for example in the pore area) in order to be able to determine a concentration of the biomolecules in the medium by measuring the flow along the flow paths.

The term “biomolecule” is to be interpreted broadly within the scope of the present invention and in particular encompass ligands, analytes, etc. The medium used can be any body fluid (especially blood). The system can also be used for water analysis, the food industry, pharmaceutical industry, etc. to detect certain substances. The membrane can be formed in one or more parts, so that the term “membrane” should also encompass various membranes. Likewise, the term “pore” should be interpreted broadly and refer to any opening or channel, as long as the opening/channel allows flow. In particular, the pore should not be restricted to a specific aspect ratio (length-to-diameter).

Optionally, the arrangement step comprises: Placing the membrane on the first detector section or on the second detector section; and then removing a portion of the membrane outside a detection area. For example, the membrane can first be applied over the entire area to one of the detector sections and then structured (for example cut to size) such that it is arranged only in one detection area between the first channel area and the second channel area.

Optionally, the method further comprises forming an adhesive layer that is in contact with the membrane. The adhesive layer can be brought into contact with the membrane in such a way that at least some of the pores are closed by the adhesive layer, in order to thereby increase the sensitivity of the membrane by reducing the number of pores for the flow measurement of the medium. For example, the adhesive layer can be used to selectively close (seal) some pores.

Optionally, the method further comprises attaching the bioreceptors to the membrane by means of a functionalization, the functionalization being carried out before or after the arrangement of the first detector section and the second detector section on opposite sides of the membrane. It goes without saying that the attachment should also include coupling and/or binding of the receptors. The functionalization can include, for example, at least the following functionalization steps: Activating a carboxy end group to obtain an amine-reactive intermediate; and amidizing the amine-reactive intermediate to form desired bioreceptors on the membrane.

The functionalization can take place in the same way in all regions of the membrane. However, it is also possible for different bioreceptors to be formed (or coupled or bound) in the pores during functionalization in different areas of the membrane, so that the membrane becomes sensitive to different biomolecules in different areas. In addition, the various functionalization steps can be carried out on a single membrane. However, it is also possible that the membrane has several parts or that several membranes are used for detection, which are to be functionalized differently.

Optionally, the method further comprises laminating the membrane to the first detector section and/or to the second detector section.

The first detector section and the second detector section can be connected to each another on the opposite sides of the membrane by a thermal treatment at a temperature of at least 50° C. or at least 65° C. Adequate impermeability can be achieved in this way. It is also possible to obtain an impermeable connection without a temperature treatment, for example by gluing.

Optionally, the concentration of the biomolecules in the medium can be determined by at least one of the following measurements: (i) a flow measurement through the at least one pore, (ii) an impedance measurement, and (iii) an electrokinetic measurement, in particular an electrophoresis or an electroosmosis measurement. In the simplest case, an electrical resistance measurement can be carried out which is proportional to the flow of the medium through the pore. In this way, a current strength and thus the number of charge carriers (i.e. ions in the medium) can be measured that pass through the pore per unit of time.

Optionally, the biomolecules include a prostate-specific antigen (PSA) and the bioreceptors aptamers. The aptamers used can in particular be one of the following aptamers:

a)
NH₂-C₆-CCGUCAGGUCACGGCAGCGAAGCUCUAGGCGCGGCCAGUUGC-
OH;
b)
NH₂-C₆-TTTTTAATTAAAGCTCGCCATCAAATAGCTTT-OH;
c)
NH₂-C₆-ACGCTCGGATGCCACTACAGGTTGGGGTCGGGCATGCGTCCGG
AGAAGGGCAAACGAGAGGTCACCAGCACGTCCATGAG-OH.

The present invention also relates to a detection system for biomolecules in a medium. The detection system comprises the following: a first channel area and a second channel area, into which the medium can be introduced, and a membrane which has at least one pore and separates the first channel region from the second channel region. In addition, a first electrode and a second electrode are formed along a flow direction of the medium on opposite sides of the membrane. Bioreceptors are formed or coupled or connected to or in the pore and comprise at least one of the following aptamers:

(i)
NH₂-C₆-CCGUCAGGUCACGGCAGCGAAGCUCUAGGCGCGGCCAGUUGC-
OH;
(ii)
NH₂-C₆-TTTTTAATTAAAGCTCGCCATCAAATAGCTTT-OH;
(iii)
NH₂-C₆-ACGCTCGGATGCCACTACAGGTTGGGGTCGGGCATGCGTCCGG
AGAAGGGCAAACGAGAGGTCACCAGCACGTCCATGAG-OH.

A PSA concentration in the medium can thus be measured via a resistance measurement along a flow path for the medium between the first electrode and the second electrode. In the simplest case, an electrical resistance of an electrolytic flow can be measured (by applying a voltage between the electrodes).

The at least one pore in the membrane can have a tapered or a cylindrical profile along the flow path.

Optionally, the membrane includes different receptors or different aptamers in different areas to enable simultaneous detection of different biomolecules.

Optionally, the first channel region and/or the second channel region perpendicular to the flow path has a maximum channel width of 50 microns or at most 10 microns. This makes it possible to effectively achieve a single-pore membrane by wetting the membrane (for example, if the pore density in the membrane is selected accordingly), which increases the sensitivity. The channel width can also be up to 1 mm. A lower limit is typically 1 micron for the materials used, but it could become lower if silicon or other materials are used.

Optionally, the detection system further comprises an electrolyte inlet at the second electrode and an analyte inlet at the first electrode in order to be able to introduce the medium in the analyte inlet and an electrolyte into the electrolyte inlet. As a result, an amount of the medium required for the detection can be reduced.

The present invention also relates to a use or a method for using one of the detection systems described for the detection of biomolecules in a medium, the detection being carried out by measuring an electrical variable which depends on an electrical resistance between the electrode and the second electrode.

Exemplary embodiments thus relate in particular to an electrochemical sensor for the detection of biotic and abiotic ligands (biomolecules). These include any molecular, organic and inorganic compound of any kind, environmental toxins, agricultural chemicals, hormones, proteins, antibiotics, neurotoxins. This also includes bacteria, viruses and parasites, which can be part of organism groups.

An advantage of exemplary embodiments lies in the fact that a cost-effective alternative to the prior art can thereby be achieved which has a higher selectivity and sensitivity. The invention further enables the integration of nanosensors in a microfluidic system which can be used as a portable mobile analyzer system for various applications, such as those mentioned above. Because of the wide range of possible uses of the exemplary embodiments, the present invention can also be used, in particular, for applications which have hitherto not been able to be analyzed, or which have only been able to be analyzed in a very complex manner.

In addition, the functionalization has a high selectivity, so that only the PSA is coupled/connected to the pore.

In particular, exemplary embodiments make it possible to significantly simplify and thus facilitate early diagnosis of prostate cancer (PCa).

### BRIEF DESCRIPTION OF THE DRAWINGS

The exemplary embodiments of the present invention will be better understood from the following detailed description and the accompanying drawings of the different embodiments, which should not be construed as limiting the disclosure to the specific exemplary embodiments but are for explanation and understanding only.

FIG. 1 shows a flow diagram for a method for producing a detection system for biomolecules according to an exemplary embodiment of the present invention.

FIG. 2 illustrates the underlying measurement principle using a membrane with at least one pore.

FIG. 3A, B show an exemplary detection system and the measurement value acquisition based on the measurement principle from FIG. 2.

FIG. 4 illustrates an exemplary functionalization of the membrane.

FIG. 5 shows a detection system according to an exemplary embodiment of the present invention.

FIG. 6A-M show a process flow for producing the detection system according to exemplary embodiments.

FIG. 7A, B show the detection systems completed with the process flow according to FIG. 6 according to further exemplary embodiments of the present invention.

### DETAILED DESCRIPTION

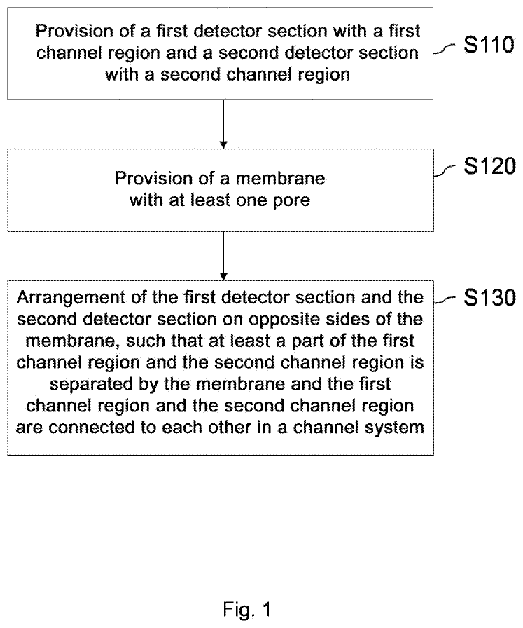

FIG. 1 shows a flowchart for a method for producing a detection system for biomolecules according to an exemplary embodiment of the present invention. The method comprises:

Providing S110 a first detector section having a first channel region and a second detector section having a second channel region;

Providing S120 a membrane having at least one pore; and

Arranging S130 the first detector section and the second detector section on opposite sides of the membrane, such that at least part of the first channel region and the second channel region are separated by the membrane and the first channel region and the second channel region are connected to each another to form a channel system to form a flow path for the medium through the at least one pore of the membrane.

It is understood that this list does not imply any order. The production steps mentioned can be carried out independently of one another or in parallel. The membrane bioreceptors are formed on the membrane along the flow path in order to determine a concentration of the biomolecules in the medium by measuring the flow (for example the resistance) along the flow path.

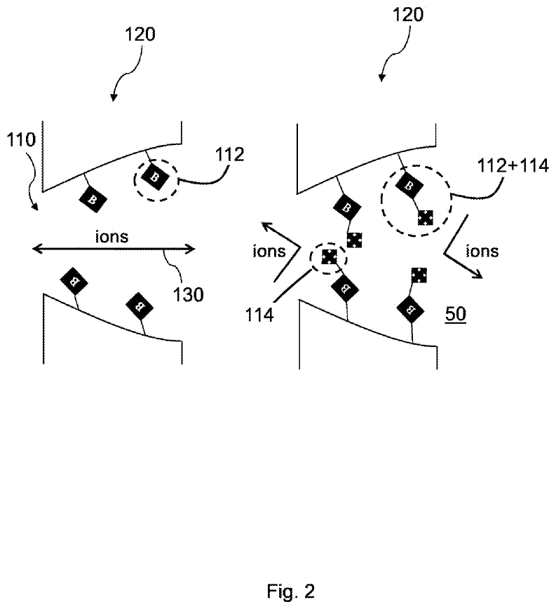

FIG. 2 illustrates the basic measurement principle using the membrane 120 with at least one pore 110. This pore 110 is, for example, a nanochannel with a (maximum) diameter of less than 1 micron (can be, for example, only a few nanometers or less than 100 nm). The membrane 120 is, for example, a single-pore plastic film.

A cross-sectional view through the pore 110 is shown on the left-hand side of FIG. 2, wherein bioreceptors 112 are attached or formed within the pore 110. These bioreceptors 112 are designed in such a way that the molecules 114 to be detected (biomolecule or analyte molecules) adhere to or are bound to them when an ion current 130 is formed through the membrane 120 (see right side of FIG. 2). The ion current 130 can be generated, for example, by an electric field that acts on charged ions in the ion current 130. By coupling and/or binding the biomolecules 114 to the bioreceptors 112, the resistance for the ion current 130 through the pore 110 changes. This change can be measured via an electrical measurement.

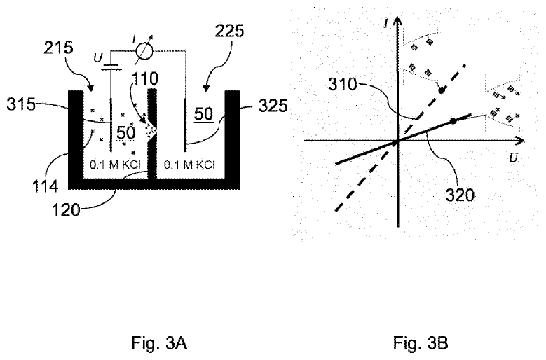

FIG. 3A shows an exemplary detection system based on the measuring principle described in the FIG. 2, wherein by way of example the structure of an electrochemical measuring cell having a single pore plastic film 120 is shown. A current-voltage characteristic (I/U characteristic) that can be measured in this way is shown in FIG. 3B, the change in the characteristic being caused by the analyte concentration in the medium 50. The binding of analyte/ligand molecules 112+114 to the functionalized nanopore 110 can therefore be determined using a (qualitative) I/U measurement.

The detection system comprises in detail a first channel region 215 and a second channel region 225 with the membrane 120 arranged between them (see FIG. 3A). Although the invention is not intended to be limited to this, it is understood that the first and second channel regions 215, 225 typically represent parts of a channel system through which the medium 50 moves. The medium can be, for example, an electrolyte (for example 0.1 M KCl, M=mol/L) with or without biomolecules. The measurement setup may also include an electrolyte container which is divided by the membrane 120 into two halves 215, 225. The membrane 120 can contain one or more nanochannels 110 as pores, which can be derivatized on the surface with covalently bound receptor molecules 112. The covalently bound receptor molecules 112 can optionally be present (almost) everywhere on the membrane 120. The receptors 112 are able to bind the biomolecules (analytes, ligands) 114 selectively and with high affinity, as a result of which the electrical resistance of the nanochannels 110 increases as a function of the concentration of the ligand molecules 114.

The medium 50 contains ions (for example as part of the electrolyte) and the biomolecules 114 to be detected, which can also be ions (but need not be). There is also a first electrode 315 in the first channel region of 215 and a second electrode 325 in the second channel region 225. By applying the voltage U between the first electrode 315 and the second electrode 325, a current I flows through the nanochannel 110 (see FIG. 2). The current I causes the biomolecules 114 to adhere to the receptor molecules 112 in the pore 110 and, as said, to change an electrical resistance as a function of a quantity of the biomolecules 114 present. The more biomolecules 114 are present, the more potentially remain in the pore 110 and thus reduce their cross-section, which is available to the ion current 130.

The change in electrical resistance can be determined by measuring the current voltage. The corresponding characteristic is shown in FIG. 3B. Two characteristic curves are shown as examples. A first characteristic curve 310 shows a measured current I as a function of the applied voltage U when there are no biomolecules 114 in the medium 50. The second characteristic curve 320 shows the current-voltage dependency in the event that a larger number of biomolecules 114 are present in the medium 50. As shown, as a result of the biomolecules 114, the current I decreases for a given voltage U, which is a consequence of the increased resistance when passing through the pore 110.

As mentioned at the beginning, a corresponding functionalization of the membrane is required, in which corresponding bioreceptors 112 are attached within the pore 110, so that the membrane is highly sensitive to certain molecules to be detected. The pore(s) themselves can also be created during the functionalization.

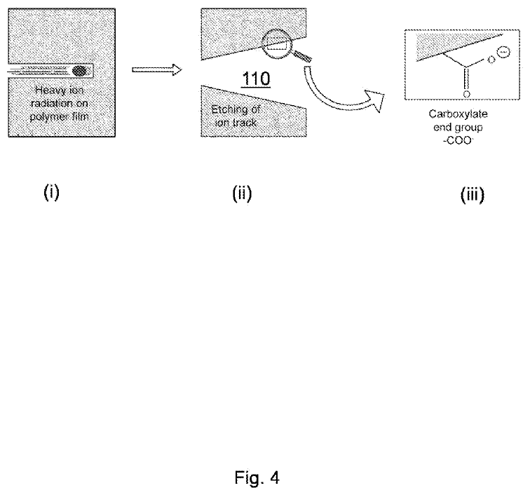

FIG. 4 illustrates an exemplary functionalization. First, a generation of the carboxylic acid/carboxylate end groups is carried out on the pore surface by irradiation and an etching process. This can be carried out, for example, in the three steps (i)-(iii) shown. In the first step (i), the membrane 120 is irradiated with heavy ions, for example, so that the ions can enter the membrane 120 and penetrate the entire membrane 120 and thus create an opening or at least break the chemical bonds there. The second step (ii) is an etching step, which leads to the ion track being widened and results in a tapered pore 110. Finally, a carboxylate end group can be formed on the surface of the pore 110 which is sensitive to the molecules 114 to be detected or which serves or can serve as an anchor point for attaching the receptors of the molecules 114 to be detected.

The surface properties can be adjusted by covalent linkage with different receptor molecules 112, such as nucleic acid aptamers (DNA/RNA). According to Ali et al. (Ali M, Nasir S, Ensinger W. 2015. Bioconjugation-induced ionic current rectification in aptamer-modified single cylindrical nanopores. Chem Commun 51: 3454-3459) the coupling can be carried out in a two-step reaction using 1-ethyl-3-(3-dimethylaminopropyl) carbodiimide (EDC) and sulfo-NHS (N-hydroxysulfosuccinimide). The reaction mechanism of linking a biological receptor 112 (e.g. an aptamer) with carboxylic acid/carboxylate groups located on the surface by EDC/NHS coupling chemistry can be realized as follows:

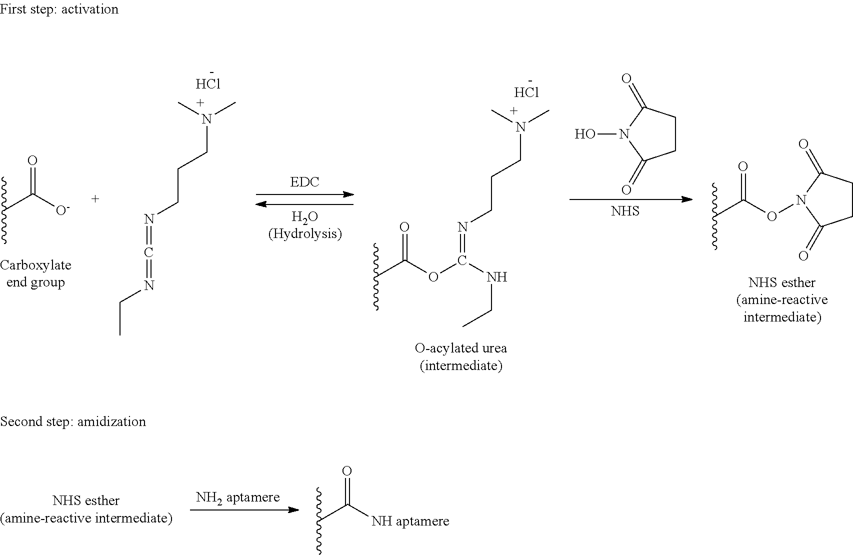

According to exemplary embodiments of the present invention, the reaction takes place in the microfluidic system, the construction and production of which is explained in more detail below.

First step (activation): Here the carboxy end groups are activated by the esterification of NHS using EDC. An O-acylated urea intermediate is initially formed, which is converted into an amine-reactive NHS ester. For this purpose, the membrane 120 is integrated into the microfluidic system. The system is then filled with a freshly prepared aqueous solution (pH 7) of 0.2 mM EDC and 0.4 mM NHS. The activation of the surface of the pores 110 is completed after one hour.

Second step (amidization): This is where the functionalization takes place with the receptor molecules 112 (aptamers), the chemical structure of which contains at least one primary amino group (—NH2). This amino group reacts with the activated carboxylic acid ester at room temperature to form an amide bond (—(C═O)—NH—). For this purpose, the microfluidic system is filled with a 0.1 mM aqueous solution of the receptor molecule 112 (aptamer) and left to stand overnight.

Successful functionalization is verified by measuring a current-voltage characteristic, since unfunctionalized and functionalized pores 110 differ at the same potential by different current strengths. This sensory principle has already been explained with FIGS. 2 and 3.

The following molecules are to be used as PSA-specific aptamers as bioreceptors 112:
1. RNA Aptamer (reference: Jeong S, Han S R, Lee Y J, Lee S W. 2010. Selection of RNA aptamers specific to active prostate-specific antigen. Biotechnol Lett 32: 379-385) Sequence (5′-3′):

NH₂-C₆-CCGUCAGGUCACGGCAGCGAAGCUCUAGGCGCGGCCAGUUGC-
OH

2. DNA Aptamer-01 (reference: Savory N, Abe K, Sode K, Ikebukuro K. 2010. Selection of DNA aptamer against prostate specific antigen using a genetic algorithm and application to sensing. Biosens Bioelectron 26: 1386-1391) Sequence (5′-3′):

NH₂-C₆-TTTTTAATTAAAGCTCGCCATCAAATAGCTTT-OH

3. DNA Aptamer-02 (reference: Duan M, Long Y, Yang C, Wu X, Sun Y, Li J, Hu X, Lin W, Han D, Zhao Y, Liu J, Ye M, Tan W. 2016. Selection and characterization of DNA aptamer for metastatic prostate cancer recognition and tissue imaging. On-cotarget 7: 36436-36446) Sequence (5′-3′):

NH₂-C₆-ACGCTCGGATGCCACTACAGGTTGGGGTCGGGCATGCGTCCGG
AGAAGGGCAAACGAGAGGTCACCAGCACGTCCATGAG-OH

The sensory properties of the functionalized (single-pore) plastic films 120 can be examined in a macro cell. For this purpose, the exemplary single-pore plastic films 120 can be used, which are manually clamped between two liquid chambers 215, 225 before each examination. The advantageous single-pore plastic films 120 are difficult to produce. In contrast, multipore plastic films can be mass-produced. However, they have a lower sensitivity compared to the individual pores.

In order to combine the advantages of both films, the wetting area of the multipore film 120 is reduced to such an extent that a single pore is still in contact with the liquid. This takes place through integration into a microsystem and thus enables the use of the detection system by untrained users.

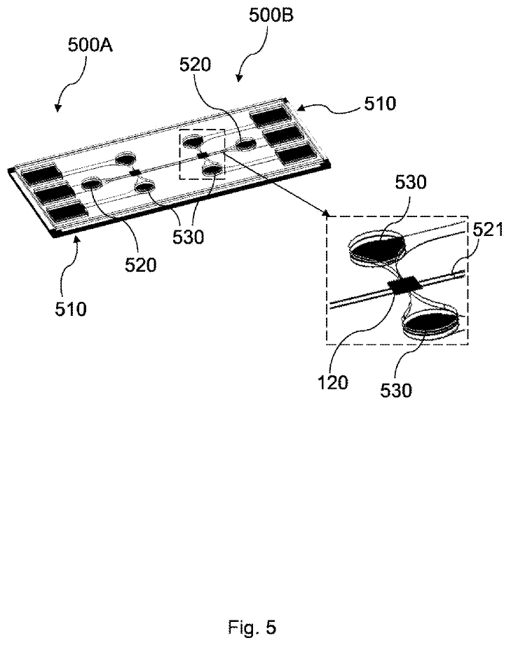

FIG. 5 shows an example of a possible detection system which comprises two detector areas 500A and 500B, which can be used for different analyzes (detection of different biomolecules). Each of the two detector areas 500A, 500B comprises three connection electrodes 510, an inlet 520 for an electrolyte and two inlets 530 for an analyte. The analyte is, for example, the medium 50 to be examined with the biomolecules 114 (analytes) and the electrolyte can be any liquid which contains ions (in order to support the electrical current) and does not falsify the result.

In the microfluidic system of FIG. 5, a multi-pore plastic film 120 is integrated between the channels for the electrolyte molecules and the biomolecules 114. All individual parts are put together by an adhesive layer. The microfluidic system can be integrated into an electronic measuring system in the form of a table device.

The second detector region 500B is shown enlarged on the right-hand side of FIG. 5, the membrane 120 being formed between two analyte inlets 530 and a channel 521 leading to the relevant electrolyte inlet 520. It goes without saying that all inlets can also be outlets. All that is required is the reversal of the direction of flow. Therefore, the analyte inlets 530 and electrolyte inlet 520 can also represent corresponding outlets. The invention is not intended to be restricted to a specific flow direction. For example, there is a fluid connection between the two analyte inlets 530 to one side of the membrane 120. The opposite side of the membrane 120 can be fluidly connected to the electrolyte inlet 520, for example. In addition, electrodes are formed in the analyte inlets 530, each of which is connected to one of the connection electrodes 510. An electrode is also formed in the electrolyte inlet 520 and is connected to one of the connection electrodes 510. A targeted application of a voltage between the electrode in the electrolyte inlet 520 and one of the electrodes in the analytical inlets 530 generates a flow of the analyte 50+114 from the respective inlet 530 to the electrolyte inlet 520.

The membrane 120 is designed, for example, horizontally and the analyte flow from one of the analyte inlets 530 takes place, for example, in the vertical direction through the membrane 120 to the channel 521, which leads to the electrolyte inlet 520. This flow can be generated either vertically downwards or vertically upwards by an applied voltage to the corresponding electrodes. The mode of operation of the detection system is illustrated further below by the representation of the production. Since there are several analyte inlets 530, different measurements can be carried out in parallel or in succession (for example for different biomolecules 114). In this way, the different analyte inlets 530 can be led to different regions of the membrane 120, which are functionalized differently, so as to allow an analysis for different biomolecules 114 in parallel.

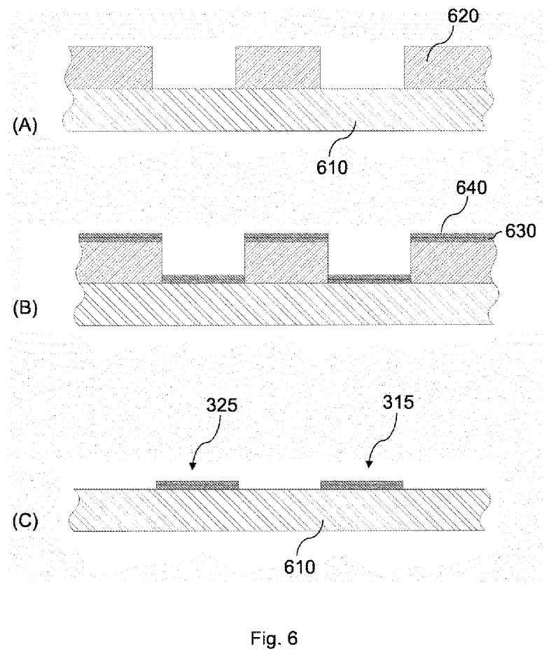

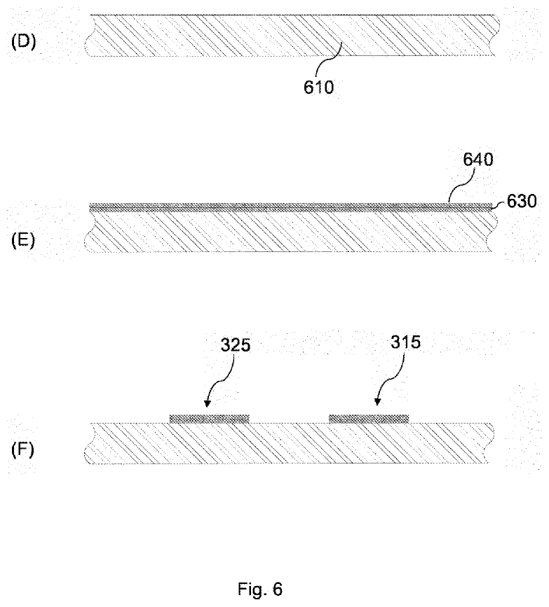

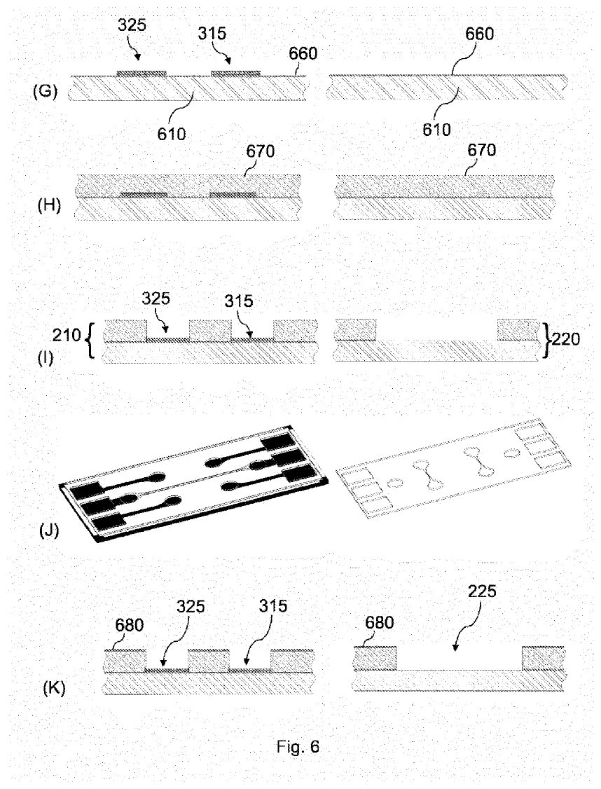

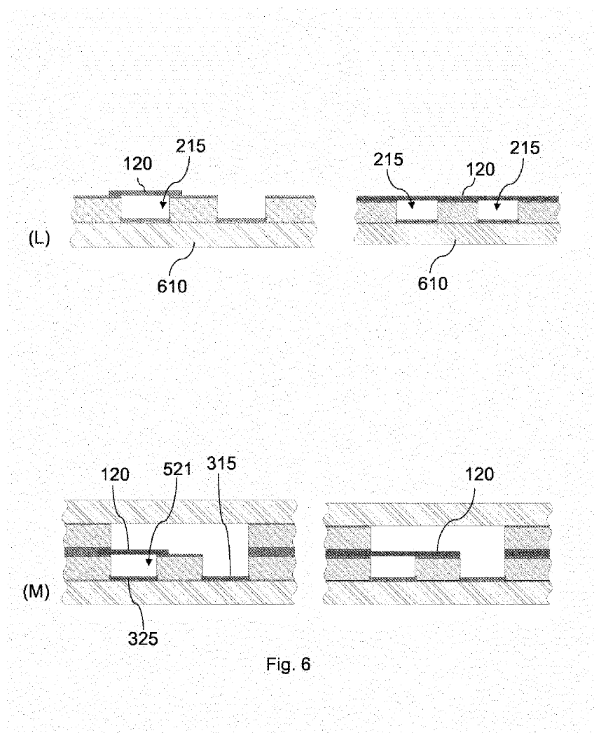

FIGS. 6A to 6M illustrate the various steps in the production of the exemplary detection system, such as that shown in FIG. 5.

FIGS. 6A to 6C first illustrate the production of the electrodes. For this purpose, a photoresist layer 620 is applied in sections on a substrate 610 (for example a glass substrate). Subsequently, an intermediate layer (for example a chrome layer) 630 and an electrode layer 640 (for example a silver layer) are deposited on the photoresist layer 620 and the exposed glass substrate sections 610. The deposition can be carried out, for example, by physical vapor deposition (PVD), for example by sputtering or vapor deposition. Finally, the photoresist layer 620 is removed together with the exemplary chrome and silver layers formed thereon, so that, as shown in FIG. 6C, the glass substrate 610 with structured electrodes 315, 325 is formed. The electrodes can represent, for example, the first electrode 315 and/or the second electrode 325.

FIGS. 6D to 6F illustrate another way to form electrodes. Here, too, an exemplary intermediate layer 630 (for example a chrome layer) is first applied to a substrate 610 and an electrode layer 640 is applied thereon, which in this exemplary embodiment can comprise gold. The layers can be applied to the substrate 610 in areas. A structuring is then carried out, i.e. the exemplary gold and chrome layers 630, 640 are removed in different areas, so that in turn only the electrodes 315, 325 remain.

The electrode structure produced in this way can also be seen in FIG. 5, where the various connection electrodes 510 are connected to electrodes in the corresponding inlets 520, 530 for the electrolyte and the analyte. The electrodes can in turn represent the first electrode 315 and/or the second electrode 325.

FIGS. 6G to 6M illustrate the production of the detection system. According to exemplary embodiments, a first detector section 210 and a second detector section 220 are created (see FIG. 6I), which are then brought together to form the detection system. For this purpose, channel structures are formed in the first detector section 210 and in the second detector section 220, which finally represent the channels for the analyte and/or the electrolyte.

In FIG. 6G, the substrate 610 with the first electrode 315 and second electrode 325 formed thereon can first be seen as an example on the left-hand side, how it can be produced with the steps from FIGS. 6A to 6F. This can later become the first detector section 210. The second detector section 220 is produced on the right-hand side in FIG. 6G, the substrate 610 again being shown first. Next, an adhesive medium layer 660 is formed on the portions shown. The adhesive medium layer 660 may have a titanium material, for example, and may have an exemplary thickness of 0.5 microns.

In the production step from FIG. 6H, a mask layer 670 (for example dry resist made of epoxy) is applied to the structures from FIG. 6G.

In the subsequent production step from FIG. 6I, the mask layer 670 is structured, the mask layer 670 being removed at the locations of the electrodes 315, 325 in the first detector section 210 and in a central region of the second detector section 220. As a result, the first electrode 315 and the second electrode 325 in the first detector section 210 and the substrate 610 in the second detector section 220 are exposed. The properties of the resulting channels (especially the hydrophilicity) can be modified by a coating.

The result is shown in the spatial representation in FIG. 6J. A plurality of electrodes is thus formed in the first detector section 210, as is shown, for example, in FIG. 5.

In FIG. 6K, an adhesive layer 680 (for example an adhesive medium layer or laminate layer, in particular a dry epoxy laminate layer) is applied to the structures from FIG. 6I as an example.

In the following step (see FIG. 6L), the membrane 120 is applied to the structures produced from FIG. 6K. According to embodiments, the membrane 120 may be applied, for example, over the full area and thermally laminated (at about T=65° C.); see right side of FIG. 6L. It needs to be also applied only partially (see left side of FIG. 6L), wherein the multipore membrane 120 is positioned between the channels or channel ranges and is subsequently thermally laminated.

If the membrane 120 is applied over the entire area to the structures of FIG. 6K (see right-hand side of FIG. 6L), the membrane 120 is subsequently structured or removed, for example at those sections which subsequently should connect the first detector section 210 and the second detector section 220 (see FIG. 6M) between the channel regions (e.g. the first and second channel regions 215, 225). Finally, the first detector section 210 and the second detector section 220 are placed on top of one another, so that the (structured) membrane 120 is arranged between the first detector section 210 and the second detector section 220. Finally, the detector produced can be laminated in order to connect all layers to one another and seal them impermeably.

The channel regions 215, 225 shown in FIG. 6M represent, for example, the fluid connection between the analyte inlet 530, through the membrane 120, via the channel 521 to the electrolyte inlet 520 (see FIG. 5). The first electrode 315 is, for example, formed below the analyte inlet 530 in FIG. 5, and the second electrode 325 is, for example, the middle electrode, which is led to the electrolyte inlet 520 via the channel 521.

According to exemplary embodiments, the above-described functionalization of the membrane 120 takes place (for example during the production step from FIG. 6M).

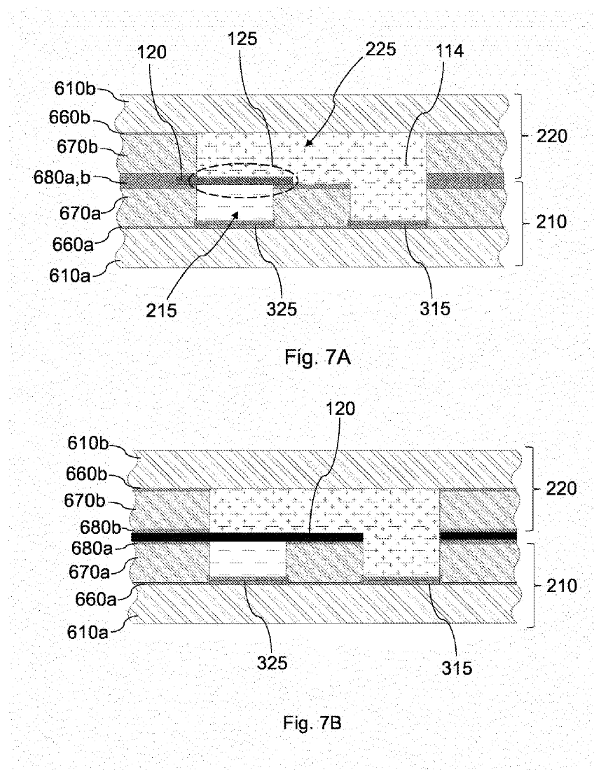

FIGS. 7A, B show completed detection systems according to exemplary embodiments of the present invention.

The exemplary embodiment in FIG. 7A shows a detection system in which the membrane 120 is formed between the first detector section 210 and the second detector section 220, specifically (for example to more than 50% or to more than 80%) only in one detection area 125, where it separates the first channel area 215 and the second channel area 225 (except for support surfaces for fixation).

As described with FIG. 6, both the first detector section 210 and the second detector section 220 each comprise a substrate 610a, 610b, between which all further layers are formed. The second detector section 220 can also be produced without a substrate. Starting with the first detector section 210, an adhesive medium layer 660a is first formed on the corresponding substrate 610a (see FIGS. 6G-6I), and a mask layer 670a is formed on the adhesive medium layer and an adhesive layer 680a is formed thereon. Below the substrate 610b of the second detector section 220, an adhesive medium layer 660b is in turn first applied, including a mask layer 670b, to which the adhesive layer 680b is in turn applied.

In addition, the first electrode 315 and the second electrode 325 are formed on the substrate 610a of the first detector section 210 (see FIGS. 6A-C). Accordingly, a flow path 130 is formed through the membrane 120 between the first electrode 315 and the second electrode 325 which triggers a current when a voltage is applied between the first electrode 315 and the second electrode 325, the resistance of which through the pore (not shown in FIG. 7A) can be measured and can be used to determine the concentration of the biomolecules 114 in the medium 50.

The exemplary embodiment of FIG. 7B differs from the exemplary embodiment of FIG. 7A only in that the membrane 120 was removed (when it was arranged) between the first detector section 210 and the second detector section 220 (essentially) only at the point where the flow path 130, starting from the first electrode 315, leaves the first detector section 210 toward the membrane 120 and enters the second detector section 220. Otherwise, the membrane 120, in particular between the adhesive layers 680a, b, which was formed as part of the first detector section 210 and the second detector section 220, is still present.

Thus, in the exemplary embodiment in FIG. 7B, the first adhesive layer 680a is at least partially or predominantly separated from the second adhesive layer 680b by the membrane 120—in particular also outside the detection area 125.

Advantageous aspects of exemplary embodiments of the present invention relate in particular to the following:
The large-/full-surface pore plastic film 120 is integrated in a lab-on-chip system between two fluid channels 215, 225 (for example in a batch process).
The film 120 is removed in the region of the fluid channels 215, 225 by laser cutting, xurography or etching (see FIGS. 6L, 6M on the right).
The pore film 120 used comprises conical pores 110 with a reproducible geometry.
The adhesive layer 680 serves both to integrate the pore 110 and to dysfunctionalize (close) the pores. Statistically speaking, only one pore can be in contact with the electrolyte. The high sensitivity of single-pore plastic films 120 is achieved with the help of multi-pore plastic films.
The functionalization of the pores 110 is carried out after the chip production, but can also be carried out before the functionalization.

Functionalization after chip production has the following advantages over pre-functionalized pores:
Only small amounts of receptor molecules 112 are required for the functionalization of the multipore plastic films 120 after the integration.
By integrating previously functionalized pores, it is possible to contaminate or clog pores.

Exemplary embodiments also offer the following advantages:
The new system has the potential to be expanded to a Micro Total Analysis System (μTAS). This enables the simultaneous detection of several ligand molecules 114.
The sensitivity of the microsystem is comparable to the single-pore measurements.
A conventional adhesive layer is a liquid UV adhesive. This leads to the clogging of the pores and is therefore not suitable for the integration of pores or functionalized pores. With the help of these conventional methods, the impermeability of the system cannot be ensured. The functionalization of the film can also be destroyed by UV exposure. In contrast to this, in exemplary embodiments of the invention, the multipore plastic film 120 is thermally integrated (at T=65° C.).
The multipore plastic films used can be functionalized both after and before integration. A yield of 100% was achieved with this method.

A channel width of 50 microns can be used, which corresponds to a wetting area of 2,500 μm². The wetting area can be further reduced to 100 μm². In conventional processes, only a wetting area of 31,416 μm² has been achieved.

The functional principle described so far is based on a voltametric method. Other measuring principles are used in further exemplary embodiments. These are for example:

(i) Flow measurement through the pore 110;

(ii) Impedance measurements; and

(iii) Electrokinetic measurements (electrophoresis, electroosmosis, etc.).

Ultimately, however, these measuring principles also measure a resistance which impedes the flow of the biomolecules 114 through the pore 110. Only the measured variable changes: in (i) the flow velocity of the medium 50; in (ii) an electrical impedance; in (iii) an electrokinematic quantity.

In comparison to current methods, which detect the respective analyte/ligand molecules in a complex manner, exemplary embodiments of the present invention enable a concentration measurement with higher selectivity and sensitivity compared to the analysis methods currently available. Different ligands in biotic and abiotic systems can be detected with this. These include the following groups of organisms and their components:

Low molecular weight organic and inorganic compounds of any kind

Environmental toxins

Agrochemicals

Hormones

Proteins

Antibiotics

Neurotoxins

Bacteria

Viruses

Parasites

The integration of the nanosensors into a mass-producible lab-on-chip system is made possible by this invention, which can be used as a compact, portable analysis system for the above-mentioned applications. This enables the measurement to be carried out within a few minutes, which can be life-saving in selected cases. The detection system can be used as a single-use microfluidic system so that it is used once for each individual test. The system can therefore be produced in large numbers.

The features of the invention disclosed in the description, the claims and the figures may be essential for the realization of the invention either individually or in any combination.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### LIST OF REFERENCE SIGNS

50 Medium
110 Pore
112 Bioreceptors
114 Biomolecules
120 Membrane
125 Detection range
130 Flow path
210, 220 Detector sections
215, 225 Channel regions
310, 320 Voltage characteristics
315 First electrode
325 Second electrode
500A, 500B Detector areas
510 Connection electrodes
520 Electrolyte inlet
521 Channel
530 Analyte inlets
610 Substrate
620 Photoresist layer
630 Intermediate layer (e.g. made of chrome)
640 Electrode layer (e.g. made of silver or gold)
660 Adhesive medium layer
670 Mask layer
680 Adhesive layer

1-15. (canceled)

16. A method for producing a detection system for biomolecules in a medium, the method comprising:
providing a first detector section with a first channel region and a second detector section with a second channel region;
providing a membrane with at least one pore;
arranging the first detector section and the second detector section on opposite sides of the membrane, so that at least part of the first channel region and the second channel region are separated by the membrane and the first channel region and the second channel region are connected to each another to form a channel system in order to form a flow path for the medium through the at least one pore of the membrane; and
bioreceptors are arranged on the membrane along the flow path through the membrane, wherein the bioreceptors are configured to determine a concentration of the biomolecules in the medium by measuring the flow along the flow path.

17. The method of claim 16, wherein the arranging step comprises:
arranging the membrane on the first detector section or on the second detector section; and then
removing part of the membrane outside a detection region.

18. The method of claim 16, further comprising:
forming an adhesive layer in contact with the membrane, the adhesive layer being brought into contact with the membrane in such a way that at least some of the pores are closed by the adhesive layer, thereby increasing a sensitivity of the membrane by reducing a number of pores for the flow measurement of the medium.

19. The method of claim 16, further comprising:
attaching the bioreceptors to the membrane by a functionalization, the functionalization being performed before or after the arrangement of the first detector section and the second detector section on opposite sides of the membrane.

20. The method of claim 19, wherein the functionalization comprises at least the following functionalization steps:
activating a carboxy end group to obtain an amine-reactive intermediate; and
amidizing the amine-reactive intermediate to form desired bioreceptors on the membrane,
wherein the functionalization occurs in a same way in all areas of the membrane or, during the functionalization, different bioreceptors are formed in the pores in the different regions, so that the membrane becomes sensitive to different biomolecules.

21. The method of claim 16, further comprising:
laminating the membrane on the first detector section and/or on the second detector section.

22. The method of claim 16, wherein the first detector section and the second detector section are connected to each other with the opposite sides of the membrane by a thermal treatment at a temperature of at least 50° C. or at least 65° C.

23. The method of claim 16, wherein a concentration of the biomolecules in the medium is determined by at least one of the following measurements:
(i) a flow measurement through the at least one pore;
(ii) an impedance measurement; and
(iii) an electrophoresis or an electroosmosis measurement.

24. The method of claim 16, wherein the biomolecules comprise prostate-specific antigens (PSA) and the bioreceptors comprise aptamers, which are one of the following aptamers:
d)
(SEQ ID NO: 1)
NH₂-C₆-CCGUCAGGUCACGGCAGCGAAGCUCUAGGCGCGGCCAGUUGC-
OH;
e)
(SEQ ID NO: 2)
NH₂-C₆-TTTTTAATTAAAGCTCGCCATCAAATAGCTTT-OH;
f)
(SEQ ID NO: 3)
NH₂-C₆-ACGCTCGGATGCCACTACAGGTTGGGGTCGGGCATGCGTCCGG
AGAAGGGCAAACGAGAGGTCACCAGCACGTCCATGAG-OH.

25. A detection system for biomolecules in a medium, the detection system comprising:
a first channel region and a second channel region into which the medium can be introduced and which have a first electrode and a second electrode;
a membrane, which comprises at least one pore, separates the first channel region from the second channel region, and is arranged fluidly between the first electrode and the second electrode,
wherein bioreceptors are formed on or in the pore and include one of the following aptamers
(iv)
(SEQ ID NO: 1)
NH₂-C₆-CCGUCAGGUCACGGCAGCGAAGCUCUAGGCGCGGCCAGUUGC-
OH;
(v)
(SEQ ID NO: 2)
NH₂-C₆-TTTTTAATTAAAGCTCGCCATCAAATAGCTTT-OH;
(vi)
(SEQ ID NO: 3)
NH₂-C₆-ACGCTCGGATGCCACTACAGGTTGGGGTCGGGCATGCGTCCGG
AGAAGGGCAAACGAGAGGTCACCAGCACGTCCATGAG-OH,
so that a PSA concentration in the medium can be measured via a resistance measurement along a flow path for the medium between the first electrode and the second electrode.

26. The detection system according to claim 25, wherein the at least one pore in the membrane has a tapered or cylindrical profile along the flow path.

27. The detection system of claim 25, wherein the membrane in different areas comprises different receptors or aptamers to enable simultaneous detection of different biomolecules.

28. The detection system of claim 25, wherein the first channel region and/or the second channel region has a maximum channel width of at most 10 microns perpendicular to the flow path.

29. The detection system of claim 25, further comprising:
an electrolyte inlet at the second electrode and an analyte inlet at the first electrode in order to be able to introduce the medium in the analyte inlet and an electrolyte into the electrolyte inlet, in order to reduce the amount of medium required for detection.

30. A method of using a detection system to detect biomolecules, the detection system comprising a first channel region and a second channel region into which the medium can be introduced and which have a first electrode and a second electrode; a membrane, which comprises at least one pore, separates the first channel region from the second channel region, and is arranged fluidly between the first electrode and the second electrode, wherein bioreceptors are formed on or in the pore and include one of the following aptamers
(i)
(SEQ ID NO: 1)
NH₂-C₆-CCGUCAGGUCACGGCAGCGAAGCUCUAGGCGCGGCCAGUUGC-
OH;
(ii)
(SEQ ID NO: 2)
NH₂-C₆-TTTTTAATTAAAGCTCGCCATCAAATAGCTTT-OH;
(iii)
(SEQ ID NO: 3)
NH₂-C₆-ACGCTCGGATGCCACTACAGGTTGGGGTCGGGCATGCGTCCGG
AGAAGGGCAAACGAGAGGTCACCAGCACGTCCATGAG-OH,
so that a PSA concentration in the medium can be measured via a resistance measurement along a flow path for the medium between the first electrode and the second electrode, the method comprising:
detecting the biomolecules in a medium by measuring an electrical variable, which is a function of an electrical resistance between the first electrode and the second electrode.


SEQUENCE LISTING (3 sequences)

SEQ ID NO: 1 - RNA, 42 nt, Artificial Sequence
  Synthetic
ccgucaggucacggcagcgaagcucuaggcgcggccaguugc

SEQ ID NO: 2 - DNA, 32 bp, Artificial Sequence
  Synthetic
tttttaattaaagctcgccatcaaatagcttt

SEQ ID NO: 3 - DNA, 80 bp, Artificial Sequence
  Synthetic
acgctcggatgccactacaggttggggtcgggcatgcgtccggagaagggcaaacgagaggtcaccagcacgtccatgag